(19) United States
(12) Patent Application Publication
(10) Pub. No.: US 2012/0283700 A1
(43) Pub. Date: Nov. 8, 2012

(54) MULTI-METAL GUIDE WIRE COIL

(75) Inventor: Matthew Pawluk, Temecula, CA (US)

(73) Assignee: ABBOTT CARDIOVASCULAR SYSTEMS INC., Santa Clara, CA (US)

(21) Appl. No.: 13/100,498

(22) Filed: May 4, 2011

(51) Int. Cl. A61M 25/09

(52) U.S. Cl. 604/528

(57) ABSTRACT

A guide wire includes a distal flexible member, such as a helical coil, which is formed with at least one highly radiopaque component and at least one component that is less radiopaque. A first wire is wound into first coils and a second wire is wound into second coils so that the first coils alternate between the second coils to form a distal helical coil. The second wire is highly radiopaque to assist a physician in positioning the distal end of the guide wire in a patient's vasculature, such as a coronary artery.

### BACKGROUND

The invention relates to the field of intracorporeal medical devices such as guide wires for advancing intra-luminal devices including stent delivery catheters, balloon dilatation catheters, atherectomy catheters and other intra-luminal devices within a patient's body lumen.

Conventional guide wires for angioplasty, stent delivery, atherectomy and other vascular procedures usually comprise an elongated core member with one or more tapered sections near the distal end thereof and a flexible body such as a helical coil or a tubular body of polymeric material disposed about the distal portion of the core member. The flexible body may extend proximally to an intermediate portion of the guide wire. A shapable member, which may be the distal extremity of the core member or a separate shaping ribbon which is secured to the distal extremity of the core member extends through the flexible body and is secured to the distal end of the flexible body by soldering, brazing or welding which forms a rounded distal tip. Torquing means are provided on the proximal end of the core member to rotate, and thereby steer, the guide wire while it is being advanced through a patient's vascular system.

Further details of guide wires, and devices associated therewith for various interventional procedures can be found in U.S. Pat. No. 4,748,986 (Morrison et al.); U.S. Pat. No. 4,538,622 (Samson et al.): U.S. Pat. No. 5,135,503 (Abrams); U.S. Pat. No. 5,341,818 (Abrams et al.); and U.S. Pat. No. 5,345,945 (Hodgson, et al.) which are hereby incorporated herein in their entirety by reference thereto.

In a typical coronary procedure using a guide wire, a guiding catheter having a preformed distal tip is percutaneously introduced into a patient's peripheral artery, e.g., femoral or brachial artery, by means of a conventional Seldinger technique and advanced and steered therein until the distal tip of the guiding catheter is seated in the ostium of a desired coronary artery.

There are two basic techniques for advancing a guide wire into the desired location within a patient's coronary anatomy through the in-place guiding catheter. The first is a preload technique which is used primarily for over-the-wire (OTW) catheters and the second is the bare wire technique which is used primarily for rapid-exchange type catheters.

With the preload technique, a guide wire is positioned within an inner lumen of an OTW device such as a dilatation catheter or stent delivery catheter with the distal tip of the guide wire just proximal to the distal tip of the catheter and then both are advanced through the guiding catheter to the distal end thereof. The guide wire is first advanced out of the distal end of the guiding catheter into the patient's coronary vasculature until the distal end of the guide wire crosses the arterial location where the interventional procedure is to be performed, e.g., a lesion to be dilated or an arterial region where a stent is to be deployed. The catheter, which is slidably mounted onto the guide wire, is advanced out of the guiding catheter into the patient's coronary anatomy over the previously introduced guide wire until the operative portion of the intravascular device, e.g., the balloon of a dilatation or a stent delivery catheter, is properly positioned across the arterial location. Once the catheter is in position with the operative means located within the desired arterial location, the interventional procedure is performed. The catheter can then be removed from the patient over the guide wire. Usually, the guide wire is left in place for a period of time after the dilatation or stent delivery procedure is completed to ensure reaccess to the distal arterial location if it is necessary. For example, in the event of arterial blockage due to dissected lining collapse, a rapid exchange type perfusion balloon catheter such as described and claimed in U.S. Pat. No. 5,516,336 (McInnes et al), can be advanced over the in-place guide wire so that the balloon can be inflated to open up the arterial passageway and allow blood to perfuse through the distal section of the catheter to a distal location until the dissection is reattached to the arterial wall by natural healing.

With the bare wire technique, the guide wire is first advanced by itself through the guiding catheter until the distal tip of the guide wire extends beyond the arterial location where the procedure is to be performed. Then a rapid exchange type catheter, such as described in U.S. Pat. No. 5,061,273 (Yock) and the previously discussed McInnes et al. patent, which are incorporated herein by reference, is mounted onto the proximal portion of the guide wire which extends out of the proximal end of the guiding catheter and which is outside of the patient. The catheter is advanced over the guide wire, while the position of the guide wire is fixed, until the operative means on the rapid exchange type catheter is disposed within the arterial location where the procedure is to be performed. After the procedure the intravascular device may be withdrawn from the patient over the guide wire or the guide wire advanced further within the coronary anatomy for an additional procedure.

An important attribute for guide wires is having sufficient radiopacity to be visualized under a fluoroscope, allowing the surgeon to advance the guide wire to a desired intra-luminal location, particularly the distal extremity of the guide wire. Unfortunately, the most suitable materials for guide wires, such as stainless steel and NiTi alloys, exhibit relatively low radiopacity. Accordingly, various strategies have been employed to overcome this deficiency. Portions of the guide wire, usually the shapeable distal tip, are typically made from or coated with highly radiopaque metals such as platinum, iridium, gold or alloys thereof. For example, a 3 to 30 cm platinum tip coil is frequently soldered to the distal extremity of the guide wire. An obvious drawback of these prior art methods is the high expense and scarcity of highly radiopaque metals and the difficulty and expense of manufacturing products from these materials. The requirement of both a high degree of radiopacity, high strength and flexibility can present design problems.

Accordingly, there remains a need for guide wires having sufficient radiopacity to allow visualization under a fluoroscope without the extensive use of expensive radiopaque metals such as platinum, gold, iridium and the like.

### SUMMARY OF THE INVENTION

The present invention is directed to an intracorporeal device such as a guide wire having an elongate core member with a proximal core section and a distal core section and a flexible body such as a helical coil formed of metallic wire which is disposed about and secured to at least a portion of the distal core section.

In accordance with the invention, the intracorporeal product has a flexible body with multiple components, at least one expensive, highly radiopaque component and at least one less expensive component having less radiopacity than the highly radiopaque component. By using a less expensive component having a lower radiopacity, it lessens the amount of the expensive highly radiopaque material which is needed in the flexible body.

In one embodiment, the guide wire has an elongated core member that has a proximal core section and a distal core section with a flexible distal tip disposed on the distal core section. The distal tip is formed from a first wire and from a second wire, where the first wire is wound into first coils so that every other first coil alternates with a wound second coil of the second wire. In this embodiment, the first wire has a radiopacity that is less than the radiopacity of the second wire. The first wire typically is formed from a metallic material taken from the group of metallic materials consisting of tantalum, stainless steel, cobalt chromium, titanium, nickel-titanium, and alloys thereof. Generally, the first wire formed from such metallic materials is not very radiopaque and is difficult to see under x-ray or fluoroscopy. The second wire is formed from a metallic material taken from the group of metallic materials consisting of platinum, gold, silver, tungsten, palladium, iridium, and alloys thereof. While the metallic material forming the second wire is highly radiopaque, it is also very expensive and generally much more expensive than the materials forming the first wire. Thus, instead of making the entire distal coil formed from the second wire of costly radiopaque materials, the first wire and the second wire are coiled in an alternating fashion in order to reduce the overall cost of the coil and at the same time retain the flexibility of the distal tip using the first wire having the less expensive and less radiopaque material.

In another embodiment, the first wire and the second wire are wound into coils so that the coils of the first wire alternate between every two coils of the second wire. In other words, moving along from the distal end of the coil toward the proximal end, there are two coils from the second wire, one coil from the first wire, two coils from the second wire, one coil from the first wire, and so on. In this embodiment, the radiopacity of the second wire is higher than the radiopacity of the first wire, and the distal tip coil overall is more radiopaque than the previously described embodiment.

In another embodiment, the first wire and second wire are formed into coils so that the first wire has two coils with one coil from the second wire in between each two coils of the first wire. In this embodiment, the highly radiopaque material of the second wire coils alternates with every two coils of the first wire, so that in this embodiment, the distal coil is less radiopaque than the previously disclosed embodiments. Importantly, the invention is intended to reduce the amount of the expensive highly radiopaque material in the distal coil while at the same time maintaining enough radiopacity to assist the physician in placing the guide wire in various body lumens such as coronary arteries.

The distal end of the helical coil may be attached directly or indirectly to the distal end of the core member and it may also be secured to the core member at one or more proximal locations.

The flexible body of the present invention has at least adequate radiopacity and strength while being substantially cheaper to make than similar structures with helical coils formed solely of precious metals such as platinum and gold. By appropriately choosing the materials, properties can be obtained which are better than conventional products, while significantly reducing costs.

These and other advantages of the invention will become more apparent from the following detailed description of the invention when taken in conjunction with the accompanying exemplary drawings.

### BRIEF DESCRIPTION OF THE DRAWINGS

FIG. 1 is a schematic elevational view, partially in section, of a guide wire which embodies features of the invention.

FIG. 2 is a transverse cross-sectional view of the guide wire shown in FIG. 1 taken along the lines 2-2.

FIG. 3 is an enlarged longitudinal cross-sectional view of the guide wire shown in FIG. 1 within the circle 3.

FIG. 4 is an enlarged longitudinal cross-sectional view of the guide wire having two second coils alternating with one first coil.

### DETAILED DESCRIPTION OF THE PREFERRED EMBODIMENTS

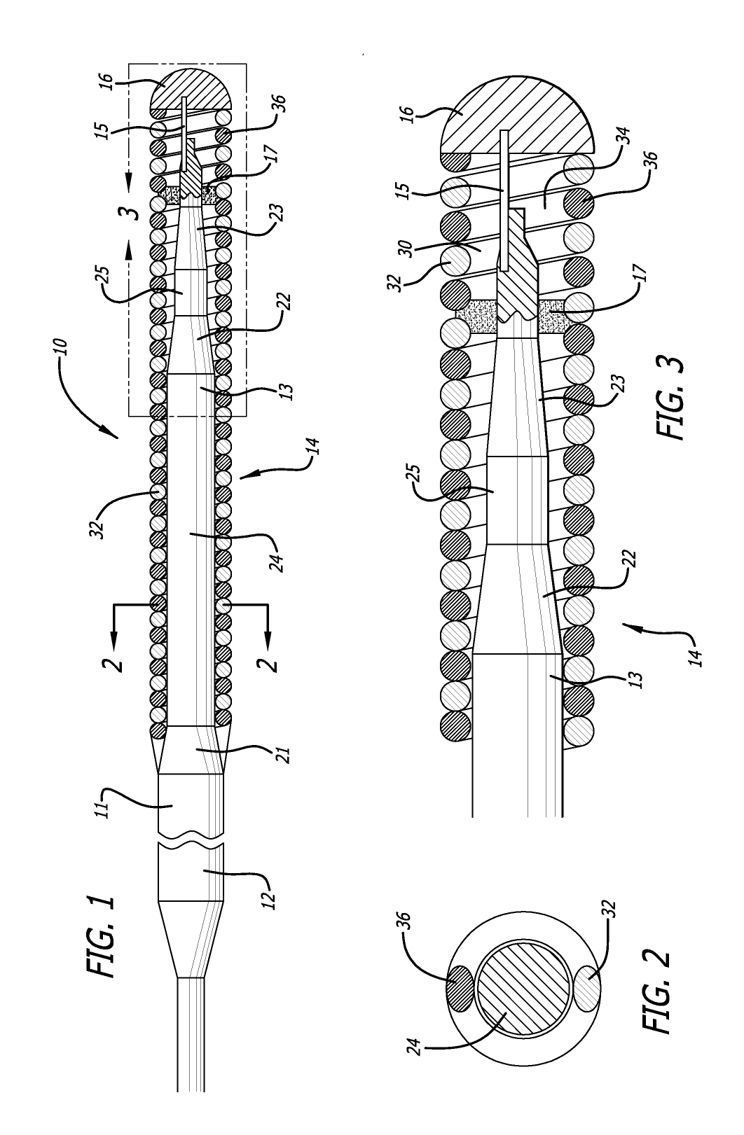

FIGS. 1-3 illustrate a guide wire 10 having features of the invention that generally include an elongated core member 11, with a proximal core section 12 and a distal core section 13 and a distal, highly flexible helical coil 14 disposed about and secured to the distal extremity of the core member. A shaping ribbon 15 (optional) extends from the distal end of the core member 11 and is secured to the mass of solder or weldment forming the rounded distal tip 16 of the guide wire. The proximal end of the shaping ribbon 15 is secured to the distal end of the core member 11 by suitable means such as solder, brazement, weldment or adhesive. The coil 14 will be secured to the core member 11 differently depending on the length of the coil. For example, a 3.0 cm long coil 14 would be secured between distal tip 16 and mass 17 which may be solder, brazement, weldment or adhesive. In another embodiment, the coil 14 would be 30 cm long and have two radiopaque coils, one 3 cm long and another 27 cm long. The 3 cm long coil would be attached between tip 16 and mass 17 while the 27 cm long coil would be secured between a first taper 21 and mass 17.

The core member 11 of the guide wire 10, as shown in FIG. 1, generally may have conventional features with conventional dimensions. The proximal core section has a relatively constant or uniform transverse cross-sectional dimensions and the distal core section 13 has a first taper 21, a second taper 22 and a third taper 23 which taper in the distal direction to smaller transverse cross-sectional dimensions. An first intermediate uniform dimensioned core portion 24 extends between the first and second tapers 21 and 22 and a second intermediate uniform dimensioned core portion 25 extends between the second taper 22 and the third taper 23.

Generally, the overall length of the guide wire may range from about 80 to about 320 cm, preferably about 160 to about 200 for coronary use. As is know in the art, there are different length guide wires for endovascular and peripheral use. Typically, commercial guide wire products of the invention will come in standard lengths of 175, 190 and 300 cm. The distal section of the guide wire is about 1 to about 10 cm, preferably about 2 to about 5 cm in length, the intermediate section is about 15 to about 50, preferably about 25 to about 40 cm in length. The outer diameter of the guide wire may vary depending upon use, but typically is about 0.008 to about 0.035 inch (0.2-0.9 mm). The lengths and diameters of the tapers may vary as well as the number of tapers and their shapes. The composite wire forming the proximal and distal coils will typically have a diameter of about 0.002 to about 0.006 inch (0.051-0.15 mm). A 0.002 inch diameter composite wire is typically used for forming a coil of about 0.010 to about 0.014 inch (0.25-0.36 mm) in diameter, a 0.0025 inch (0.063 mm) wire for a coil with an OD of 0.0014 inch and a wire of 0.0055 inch (0.14 mm) for larger OD coils. To the extent not otherwise described herein, the dimensions, constructions and materials of the guide wire may be conventional.

Referring to FIGS. 1-4, the guide wire 10 includes a highly flexible helical coil 14 comprised of two different metals. In one embodiment, the helical coil 14 has a first wire 30 wound into helical first coils 32 and a second wire 34 wound into helical second coils 36. The first wire is formed from a metallic material take from the group of metallic materials that include any of stainless steel, tantalum, cobalt-chromium, titanium, nickel-titanium, and alloys thereof. The first wire 30 has a substantially lower radiopacity than the second wire 34, but the material of the first wire generally is much less expensive than that of the second wire. With respect to the second wire 34, it is formed from a metallic material taken from the group of metallic materials including platinum, gold, palladium, iridium, silver, bismuth, and tungsten, and alloys thereof. The second wire metallic materials are highly radiopaque and can be seen under x-ray and fluoroscopy for the purpose of positioning the distal end of the guide wire. The second wire 34 is substantially more radiopaque than the first wire 30, however second wire 34 is made from materials that are substantially higher cost than the metallic materials forming the first wire. In one aspect of the invention, in order to reduce the cost of forming the helical coil 14, the first wire 30 and the second wire 34 are wound into helical coils in an alternating fashion, with first coils 32 alternating with second coils 36. In this embodiment, the radiopacity of the helical coil 14 will be less than if the helical coil were made solely of the second wire metallic materials. In other words, if helical coil 14 were formed solely of platinum, it would be very expensive yet highly radiopaque. In the present invention, the helical coil 14 would have half the amount of platinum coils since the second wire 34 formed of the platinum material alternates with the first coils 32 of the first wire 30 which is made of a different material such as stainless steel.

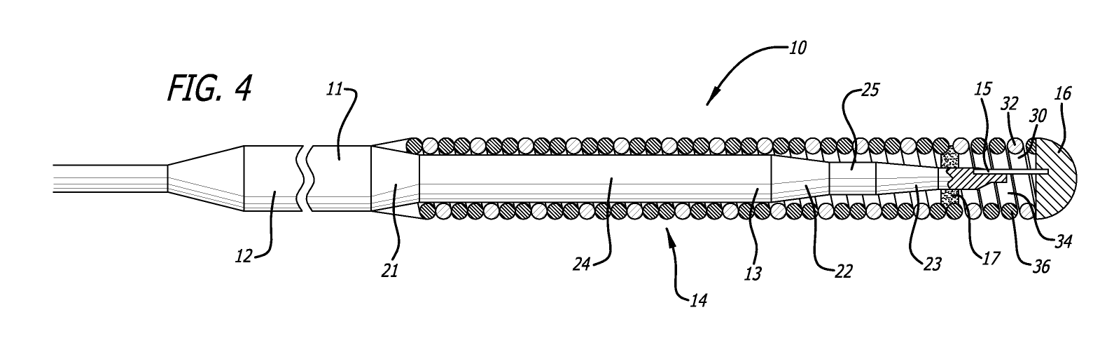

In another embodiment, as shown in FIG. 4, the helical coil 14 includes a first wire 30 having first coils 32 and a second wire 34 having second coils 36. In this embodiment, the first wire 30 is wound into the first coils 32 and the second wire is wound into the second coils 36 so that there is one first coil 32 wound in between every two second coils 36. The radiopacity of this embodiment of helical coil 14 is less than if the helical coil were made purely of just the second wire material, but it has a higher radiopacity than the previous embodiment in FIG. 3 because there are twice as many second coils 36 in this embodiment. Thus, the helical coil of this embodiment is still highly radiopaque and is less expensive than if the helical coil were made solely of the more expensive radiopaque materials. Other combinations are contemplated, such as three second coils alternating with every one first coil, two first coils alternating with every two second coils, and three first coils alternating with every one second coil.

With respect to any of the embodiment disclosed herein, the pitch and dimensions of the first wire 30 and the second wire 34 coils can vary as needed. In fact, the outer diameters of the first coils 34 and the second coils 36 do not need to be the same, although it is preferable that they be close in their dimensions for a multi-filer coil winder to be able to wind the first wire 30 and the second wire 34 into coils at the same time.

The metallic material forming the first wire 30 typically is more solderable than the metallic material forming the second wire 34, so the first wire coils 32 can be soldered and assist in holding the second coils 36 together. As is known in the art, helical coils may have a polymer coating on the wires, either on the outside or the inside of the coil, or any variation thereof. Such polymer coatings assist in holding the coils in place without unduly decreasing the flexibility of the helical coil.

Although individual features of embodiments of the invention may be shown in some of the drawings and not in others, those skilled in the art will recognize that individual features of one embodiment of the invention can be combined with any or all the features of another embodiment. Various modification may be made to the invention without departing from the scope thereof.

What is claimed:

1. A guide wire, comprising:
an elongated core member having a proximal core section and a distal core section;
a flexible distal coil disposed on the distal core section formed of a first wire and a second wire, the first wire being wound into first coils so that every other first coil alternates with a wound second coil of the second wire; and
the first wire has a radiopacity that is less than a radiopacity of the second wire.

2. The guide wire of claim 1, wherein the second wire is formed from a metallic material taken from the group of metallic materials consisting of platinum, gold, palladium, iridium, tungsten, silver and bismuth, and alloys thereof.

3. The guide wire of claim 1, wherein the first wire is formed of a metallic alloy having a radiopacity that is substantially less than the radiopacity of the second wire.

4. The guide wire of claim 3, wherein the first wire is formed from a metallic material taken from the group of metallic materials consisting of tantalum, stainless steel, cobalt-chromium, titanium, nickel-titanium and alloys thereof.

5. The guide wire of claim 1, wherein the first wire and the second wire are helically wound to form the flexible distal coil.

6. The guide wire of claim 1, wherein the first wire is wound into the first coils and the second wire is wound into the second coils so that there is a first coil wound between every two second coils.

7. The guide wire of claim 1, wherein the first wire is wound into the first coils and the second wire is wound into the second coils so that there is a first coil wound between every three second coils.

8. The guide wire of claim 1, wherein the first wire has a first diameter and the second wire has a second diameter, the first diameter being equal to the second diameter.

9. The guide wire of claim 1, wherein the first wire has a first diameter and the second wire has a second diameter, the first diameter being greater than the second diameter.

10. The guide wire of claim 1, wherein the first wire has a first diameter and the second wire has a second diameter, the first diameter being less than the second diameter.

11. A guide wire, comprising:
an elongated core member having a proximal core section and a distal core section;
a flexible body disposed on the distal core section formed of a helically wound first wire having a first radiopacity, and a helically wound second wire having a second radiopacity;
wherein the second wire being formed from a material selected from the group consisting of platinum, gold, palladium, iridium, and alloys thereof; and
wherein the first wire being formed from a material selected from the group consisting of tantalum, tungsten, stainless steel, cobalt chromium, titanium, nickel-titanium, and alloys thereof.

12. The guide wire of claim 11, wherein the flexible body includes a coil.

13. The guide wire of claim 12, wherein the first wire is formed into the coil and the second wire is formed into the coil so that the first wire has turns that alternate with turns of the second wire.

14. The guide wire of claim 11, wherein the first wire is helically wound into a first coil and the second wire is helically wound into a second coil, the first coil having turns that alternate with turns in the second wire.

15. A guide wire, comprising:
an elongated core member having a proximal core section and a distal core section;
a flexible distal coil disposed on the distal core section formed of a first wire and a second wire, the first wire being wound into first coils so that one or more first coils are wound between one or more second coils of the second wire; and
the first wire has a radiopacity that is less than a radiopacity of the second wire.

16. The guide wire of claim 15, wherein the second wire is formed from a metallic material taken from the group of metallic materials consisting of platinum, gold, palladium, iridium, tungsten, silver and bismuth, and alloys thereof.

17. The guide wire of claim 15, wherein the first wire is formed of a metallic alloy having a radiopacity that is substantially less than the radiopacity of the second wire.

18. The guide wire of claim 17, wherein the first wire is formed from a metallic material taken from the group of metallic materials consisting of tantalum, stainless steel, cobalt-chromium, titanium, nickel-titanium and alloys thereof.

19. The guide wire of claim 15, wherein the first wire and the second wire are helically wound to form the flexible distal coil.
